(19) United States
(12) Patent Application Publication
(10) Pub. No.: US 2006/0293748 A1
(43) Pub. Date: Dec. 28, 2006

(54) Prosthetic implant, and a method and tool for the insertion of same

(75) Inventors: Gerald John Alexander, Irvine, CA (US); Douglas Neary, Huntington Beach, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpineWorks, LLC, Huntington Beach, CA (US)

(21) Appl. No.: 11/166,634

(22) Filed: Jun. 24, 2005

(51) Int. Cl. A61F 2/44; A61F 2/46; A61B 17/88

(52) U.S. Cl. 623/17.11; 623/908

(57) ABSTRACT

Disclosed herein is a prosthetic implant, a tool assembly for positioning the prosthetic implant, and a method of placing a prosthetic implant. The prosthetic implant includes an anterior end and an opposite, smaller, posterior end. Left and right side portions attach the anterior end to the posterior end. Each of the left and right side portions includes serrations on the top and bottom. The serrations on the top are directed substantially opposite the direction of the serrations on the bottom. Adjacent serrations can be separated by a radius. The implant includes tooling apertures for receiving corresponding portions of a tool assembly. The tool assembly includes an inner tool and an outer tool. The inner tool can engage the implant and can seat the outer tool to the implant. The outer tool can be used to exert a rotational force to the implant to rotate the implant to a desired orientation.

### FIELD OF THE DISCLOSURE

The present disclosure relates generally to prosthetic implants and, more specifically, to an intervertebral implant used for spinal fusion.

### BACKGROUND OF THE DISCLOSURE

Prosthetic implants inserted into the spine have been useful in treating patients with damaged or degenerated invertebral discs. Typically, spinal prosthetic implants are inserted from the posterior side of the spinal column. To insert a prosthetic implant from the posterior side is challenging since the invertebral space in the spinal column is smaller in the posterior side of the spinal column versus the anterior side. Some methods of insertion require the surgeon to open the area of insertion of the spinal column by distracting between pedicle screws above and below and then subsequently compressing between the screws once the prosthetic implant is in place. Another method may include distracting on the vertebrae themselves with a thin long tipped distracter which is inserted from one side of the space in the spinal column while the prosthetic implant is inserted into the other side, such as the posterior left side versus the posterior right side of the vertebrae. These procedures take much time and do have risks associated with them. For example, distracting on the screws can loosen the screws by decreasing the purchase of the implant within the bone pedicles. This is particularly true in patients who have soft bone.

The other method described above which requires the distracting on the vertebrae themselves, requires wide exposure of the vertebrae and the distracter can slip and damage the patients nerves in the spinal column. In addition, the distracter can get in the way of the prosthetic implant being inserted into the spinal column, and the distracter requires an additional set of hands to hold the distracter during the surgical procedure.

Therefore, what is needed, is an improved prosthetic implant and method that overcomes the limitations of the currently available prosthetic implants and methods for inserting same.

### SUMMARY OF THE DISCLOSURE

Disclosed herein is a prosthetic implant that includes an anterior end, a posterior end being located opposite of the anterior end, the anterior end being larger than the posterior end, the anterior end and the posterior end being separated by a left side portion and an opposite right side portion, and the left side portion and the right side portion each having a top side and an opposite bottom side, the top and bottom sides of the left side portion and the right side portion having serrations.

Also disclosed is a method for placing a prosthetic implant, wherein the prosthetic implant includes, an anterior end, a posterior end being located opposite of the anterior end, the anterior end being larger than the posterior end, the anterior end and the posterior end being separated by a left side portion and an opposite right side portion, and the left side portion and the right side portion each having a top side and an opposite bottom side, the top and bottom sides of the left side portion and the right side portion having serrations, and wherein the anterior end includes an anterior tooling aperture and anterior rotational tooling apertures, and wherein the posterior end includes an posterior tooling aperture and posterior rotational tooling apertures, and wherein the method includes the steps of, creating a void in either the anterior or the posterior side of an area generally between two vertebrae in a spinal column; and inserting the posterior end of the prosthetic implant into the vertebrae first if the void is created from the anterior side of the vertebrae, or inserting the anterior end of the prosthetic implant into the vertebrae first if the void is created from the posterior side of the vertebrae.

Also disclosed herein is a prosthetic implant, method, and tool for placing the implant, wherein the implant is angled to provide to correct spinal curvature after placement and wherein the anterior end and the opposite posterior end are rounded to provide ease of placement into the proper location between the vertebrae. The prosthetic implant has serrated edges on the top sides and the bottom sides to allow for purchase or digging into the bone and to prevent migration out of the placed location. Further, the prosthetic implant disclosed herein is insertable into the spinal column from the posterior side of the patient as well as the anterior side of the patient. The prosthetic implant includes tooling apertures and rotational tooling apertures to allow for the placement of the prosthetic implant from the anterior side or posterior side of the spinal column. The rotational tooling apertures allow the prosthetic implant to be rotated ninety degrees, particularly when inserted from the posterior side of the spinal column.

### BRIEF DESCRIPTION OF THE DRAWINGS

The invention will now be described in greater detail with reference to the embodiments illustrated in the accompanying drawings, in which like elements bear like reference numerals, and wherein:

FIG. 1 is a prospective view of a prosthetic implant of an anterior end of the prosthetic implant according to the present disclosure;

FIG. 2 is a right side plan view of the prosthetic implant;

FIG. 3 is a left side plan view of the prosthetic implant;

FIG. 4 is a top plan view of the prosthetic implant;

FIG. 5 is a posterior end view of the prosthetic implant;

FIG. 6 is an anterior end view of the prosthetic implant;

FIG. 7 is a cut away view of the prosthetic implant of the present disclosure shown placed in the spinal column between two vertebrae;

FIG. 8 is a view taken from Section 8-8 from FIG. 7 showing two prosthetic implants placed in the spinal column between two vertebrae;

FIG. 9 illustrates a view taken from Section 9-9 from the FIG. 8 illustrating the placement of two prosthetic implants in the spinal column between two vertebrae;

FIG. 10 illustrates a side view of the outer tool;

FIG. 11 illustrates a distal end view of the outer tool;

FIG. 12 illustrates a side view of the inner tool;

FIG. 13 illustrates a distal end view of the inner tool;

FIG. 14 illustrates a side view of the tool assembly for placing the prosthetic implant;

FIG. 15 illustrates a distal end view of the tool assembly according to the present disclosure;

FIG. 16 illustrates a side views of the tool assembly and prosthetic implant while the tool assembly is engaged with the posterior end of the prosthetic implant;

FIG. 17 illustrates a prospective view of the alternative tool assembly for placing the prosthetic implant; and

FIG. 18 illustrates a side view of the alternative tool assembly for placing the prosthetic implant.

### DETAILED DESCRIPTION OF THE PREFERRED EMBODIMENTS

Disclosed herein is a prosthetic implant 100 that may be placed into a patient's spinal column from both the posterior side of the spinal column and the anterior side of the spinal column. Further, the prosthetic implant 100 disclosed herein is wedged shape to help maintain the curvature of the spinal column of the patient after placement of the prosthetic implant 100 between two vertebrae. The prosthetic implant 100 at the present disclosure further includes serrations on the top 130a-b and bottom 132a-b sides at the prosthetic implant 100 to allow purchase or digging into the bone of the vertebrae of the prosthetic implant 100 during and after placement. Further, the serrations 130 a-b, 132a-b of the prosthetic implant 100 on the top of each side are positioned in a direction that substantially opposes the direction of the serrations on the bottom of the side to help to negate any migration of the prosthetic implant 100 out of the placed location in the spinal column. Further, the prosthetic implant 100 disclosed herein includes rounded edges 136 and 134 on both posterior 104 and anterior 102 ends, respectively, to allow for the ease of insertion of the prosthetic implant 100 into a prepared location in the spinal column. A plurality of apertures 116, 118, 120, 122 and a through slot 114 allow for bone growth through the prosthetic implant 100 to allow for oseointegration of the prosthetic implant 100 to the bone at the placed location in the spinal column.

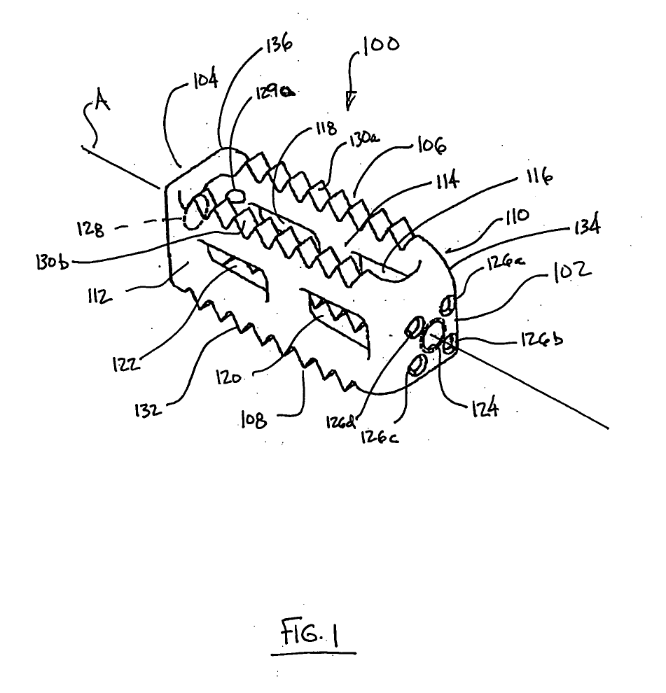

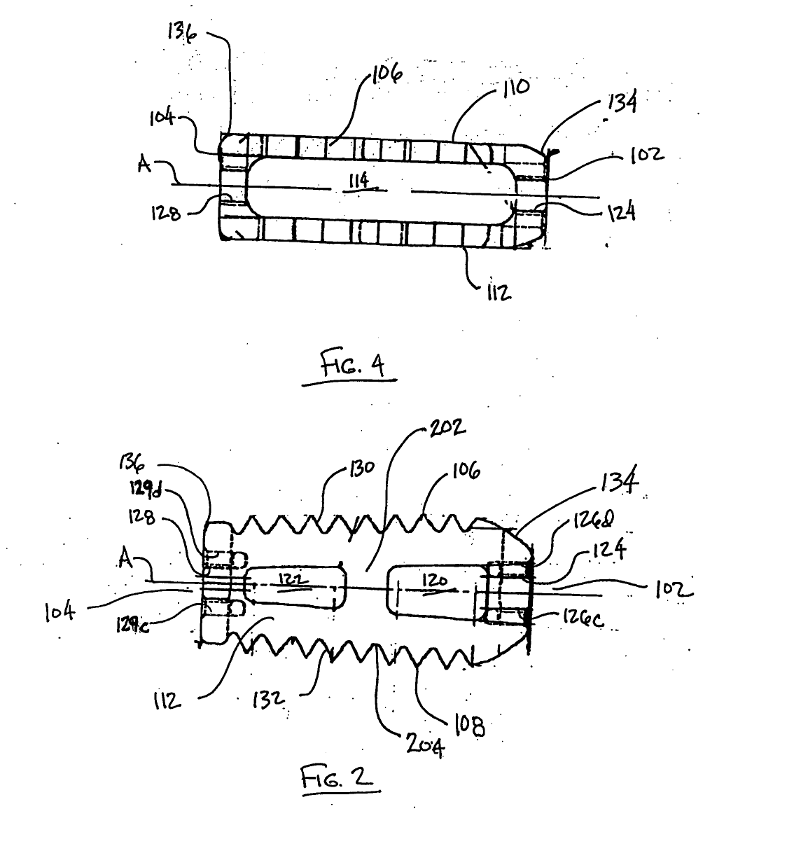

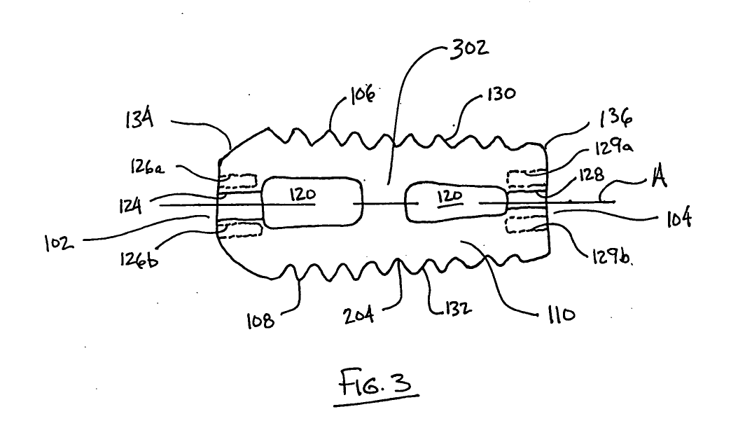

As shown in FIG. 1, a prosthetic implant 100 according to the present disclosure includes a major latitudinal axis A with an anterior end 102 at one end and a posterior end 104 at an opposite end. Further, the prosthetic implant 100 has a top side 106 and an opposite bottom side 108. As will be further described below, the top side 106 and the bottom side 108 each have two surfaces that are defined by a left side portion 110 and an opposite right side portion 112. The left side portion 110 and right side portion 112 further define a longitudinal through slot 114. The left side portion 110 further includes a left side anterior aperture 116 and a left side posterior aperture 118. The right side portion 112 further includes a right side anterior aperture 120 and a right side posterior aperture 122. The left side anterior aperture 116 and the left side posterior aperture 118 define a left side rib 302, as shown in FIG. 3. Further, the right side anterior aperture 120 and the right side posterior aperture 122 define a right side rib 202, as shown in FIG. 2.

The longitudinal through slot 114, the left side anterior side aperture 116, left side posterior aperture 118, right side anterior aperture 120, and right side posterior aperture 122 combine to provide a large surface area and volume to hold bone graft material during the surgery and placement of the prosthetic implant 100 into the spinal column. The large surface area and volume for holding the bone graft material promotes healing and oseointegration of bone growth adjacent to and through the prosthetic implant 100.

The prosthetic implant 100 of the present disclosure further includes certain features for the ease of placement of the prosthetic implant 100 into the prepared area in the spinal column. For example, the anterior end 102 includes an anterior tooling aperture 124. The anterior tooling aperture 124 may be threaded to allow a tool assembly 1000 to selectively fasten and unfasten the tool assembly 1000 to and from the prosthetic implant 100. In general, the anterior end includes a portion that is configured to engage a tool assembly, such as the tool assembly 1000. The portion can include a tooling aperture 124 as shown in FIG. 1. In other embodiments, the portion of the prosthetic implant that engages a tool assembly may be a male end rather than a female end, or may include both male and female portions.

Further, the anterior end 102 further includes one or more anterior rotational tooling apertures 126. The rotational tooling apertures 126 are configured to receive a rotational force exerted on the prosthetic implant by the tool assembly 1000. The rotational tooling apertures 126 may be any type of receiving portion and need not be apertures. For example, the rotational tooling apertures or receiving portion may be holes, indentations, pins, flanges, and the like or some combination of male and female portions configured to receive a rotational force.

In the example shown, the prosthetic implant includes four anterior rotational tooling apertures 126a-d, however, as long as at least one anterior rotational tooling aperture 126 is provided, the tool assembly 1000 would be capable of rotating the prosthetic implant 100 upon insertion of the prosthetic implant 100 at the desired depth into the prepared area in the spinal column. Four anterior rotational tooling apertures 126a-d provide means for distributing the force evenly from the tool assembly 1000 to the prosthetic implant 100 during rotation of the prosthetic implant 100 in the prepared area in the spinal column. Nevertheless, any number of rotational tooling apertures 126, 129 would suffice.

The anterior tooling aperture 124 allows the tool assembly 1000 to selectively fasten to and unfasten from the prosthetic implant 100 at the anterior end 102 to allow for insertion of the posterior end 104 first into the anterior side of the spinal column or of the anterior side of the patient. For example, in certain situations the surgeon would prefer to insert the prosthetic implant 100 into the spinal column of the patient from the anterior side of the patient, and in this example, as provided by the present disclosure, the tool assembly 1000 would be selectively fastened to the anterior end 102 of the prosthetic implant 100 with the posterior end 104 being inserted into the anterior side of the patient or the anterior side of the spinal column with the posterior end 104 of the prosthetic implant 100 being inserted first into the prepared area at the anterior side of the spinal column.

The posterior end 104 further includes a posterior tooling aperture 128, as best shown in FIG. 5. Similar to the anterior tooling aperture 124, the posterior tooling aperture 128 includes means to allow the tool assembly 1000 to be selectively fastened to and unfastened from the posterior end 104 of the prosthetic implant 100. In the example shown, the posterior tooling aperture 128 is threaded to allow the tool assembly 1000 to be selectively fastened to and unfastened from the posterior end 104 by allowing the tool assembly 1000 to be threaded into or out of the posterior tooling aperture 128. The posterior end 104 further includes means to rotate the prosthetic implant 100 upon insertion into the prepared area in the spinal column, if desired. In this example, the posterior end 104 includes posterior rotational tooling apertures 129a-d, and similar anterior rotational tooling apertures 126a-d, the posterior rotational tooling apertures 129a-d could comprise any number greater than two so as to allow the tool assembly 1000 to rotate the prosthetic implant 100 upon placement into the prepared area in the posterior side of the spinal column. In the example shown, four posterior rotational tooling apertures 129a-d are provided to allow for the distribution of the rotational forces from the tool assembly 1000 to the prosthetic implant 100.

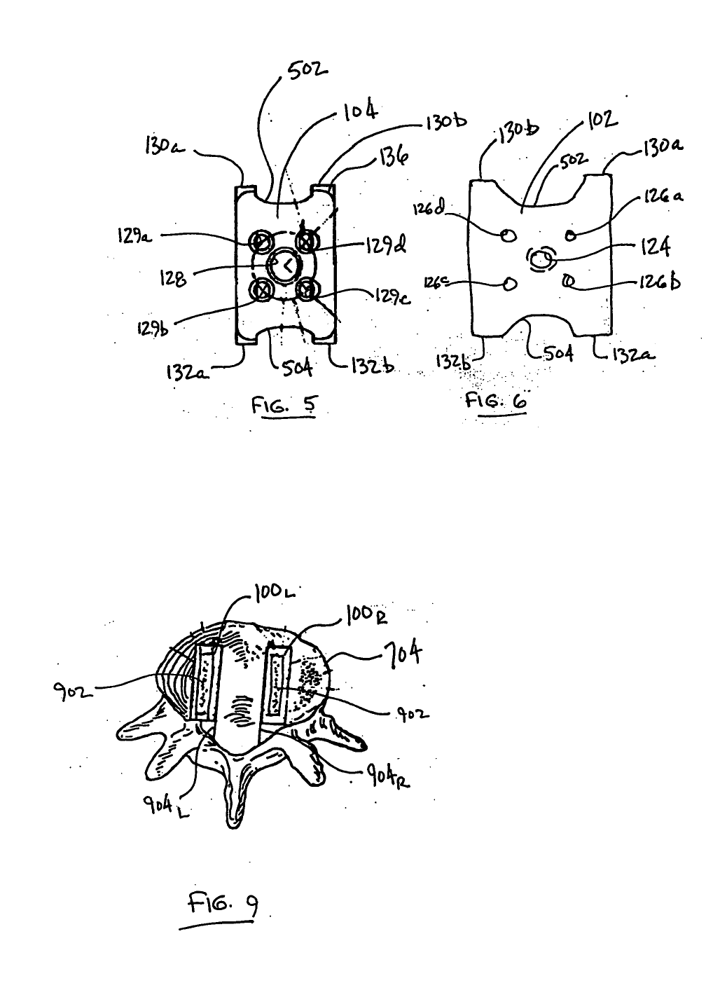

The prosthetic implant 100 further includes top side serrations 130a and 130b with top side serrations 130a being located at the top side of the left side portion 110. Further, bottom side serrations 132a are located at the bottom side of left side portion 110. Top side serrations 130b are located on the top side of right side portion 112 and likewise bottom side serrations 132b are located on the bottom side of right side portion 112. The top side serrations 130a and 130b further define a top channel 502 as shown in FIGS. 5 and 6. Likewise, bottom side serrations 132a and 132b further define a bottom channel 504. The top channel 502 and the bottom channel 504 provide a large surface area and volume for holding the bone graft material which promotes healing and osteointegration of bone growth adjacent to and within the prosthetic implant 100.

The anterior end 102 further includes anterior end rounded edges 134 to allow for the ease of insertion of the prosthetic implant 100 into the prepared area in the spinal column, likewise, the posterior end 104 further includes posterior end rounded edges 136 to ease with the insertion of the posterior end 104 into the prepared area in the spinal column when the posterior end 104 is inserted first into the spinal column.

The serrations 130 and 132 include a plurality of bottom radii 204 in which a radius is provided at the bottom of each serration 130, 132. The serrations 130, 132 can include a plurality of peaks. Adjacent peaks within the serrations can be separated by the bottom or lower radius. The radius between adjacent peaks can help to reduce the amount of stress at each location to remove the probability of a stress fracture in the prosthetic implant 100 at each radius 204 area.

FIGS. 7-9 illustrate the prosthetic implant 100 after placement into the spinal column. FIG. 7 illustrates the prosthetic implant 100 inserted into a prepared area that enters the posterior side of the spinal column.

In this example, as shown, an area is prepared from the posterior side of the spinal column. The spinal area can be prepared by removing some of the spinal material, which may include bone and portions of a disc. In one method, a biomedical drill bit can be used to drill a placement area between two vertebrae in the spinal column where soft tissue is located or where soft tissue typically would be located. A disc that is located between two vertebrae is typically removed. The disc can be removed, for example, manually using hand tools such as curettes.

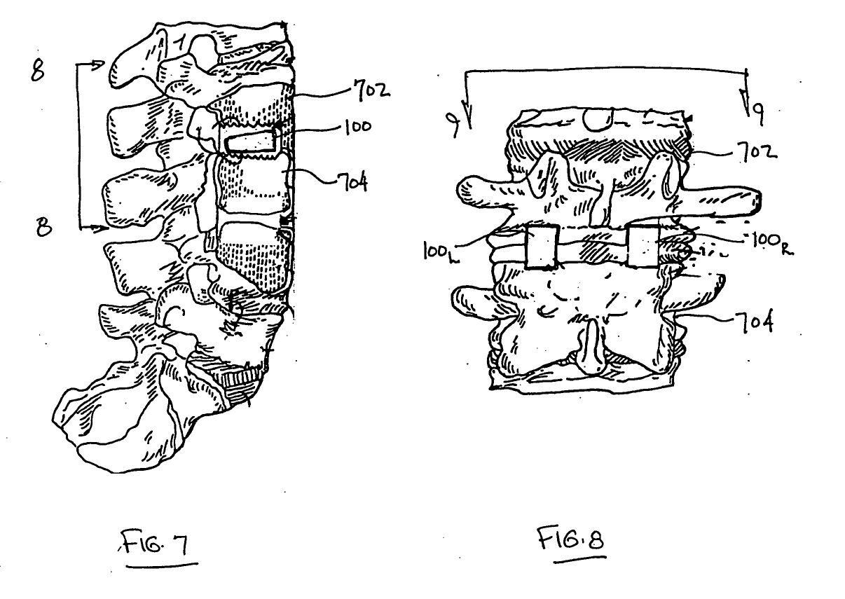

For example, if the patient has a herniated or degenerated disc between two vertebrae, the patient may require two vertebrae to be fused together by the use of one or more prosthetic implants 100 of the present disclosure. In the example shown in FIGS. 7-9, two prosthetic implants 100 are located in the fusion area between the two vertebrae. The prosthetic implant 100 is located between vertebrae 702 and vertebrae 704. As stated above, the prosthetic implant 100 is located so that the anterior end 102, which is larger than the posterior end 104, is located on the anterior side of the spinal column. The larger anterior end 102 helps to maintain the natural spinal curvature in the patient. FIG. 8 illustrates a posterior view of the spinal column taken from Section 8-8 from FIG. 7. FIG. 8 illustrates the placement of two prosthetic implants 100 located on a right side of the spinal column and a left side of the spinal column. Prosthetic implant 100ᵣ is the implant located on the right hand side of the spinal column and prosthetic implant 100ₗ is located oh the left hand side of the spinal column.

FIG. 9 illustrates a top sectional view taken from Section 9-9 from FIG. 8 looking down on two prosthetic implants 100 placed in the spinal column from the posterior side of the spinal column of the patient. Bone graft material 902 is shown located in the longitudinal through slots 114. FIG. 9 also shows passage ways 904ᵣ and 904ₗ created for the implants.

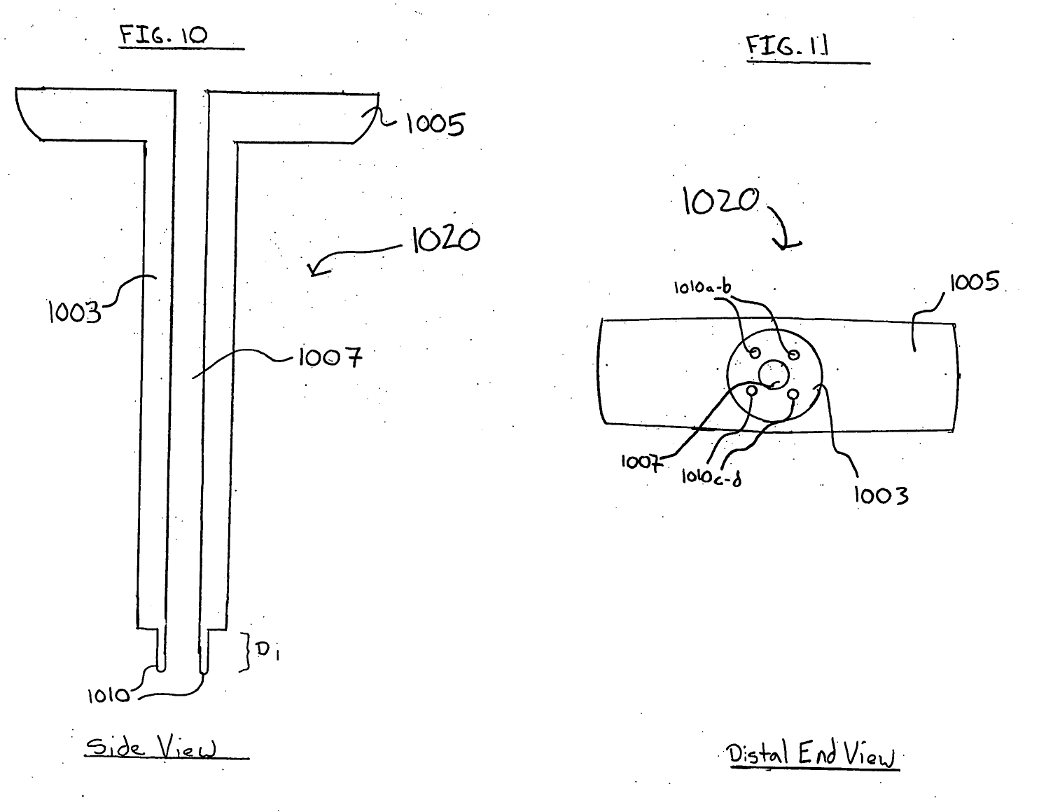

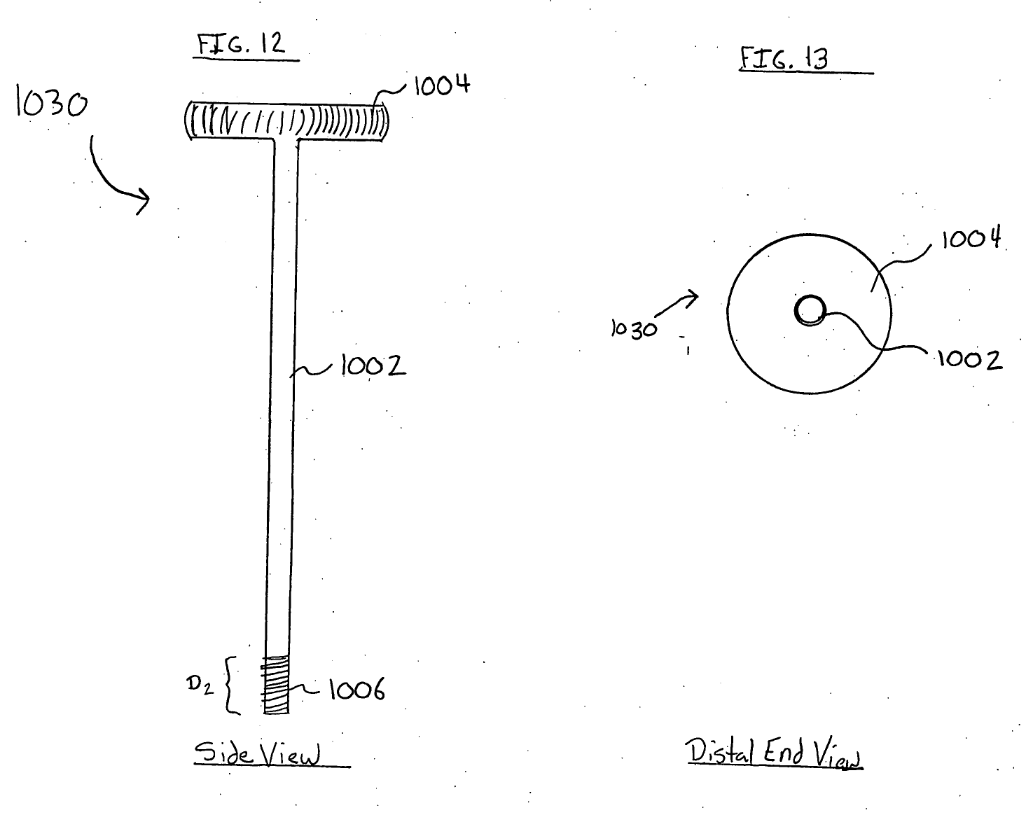

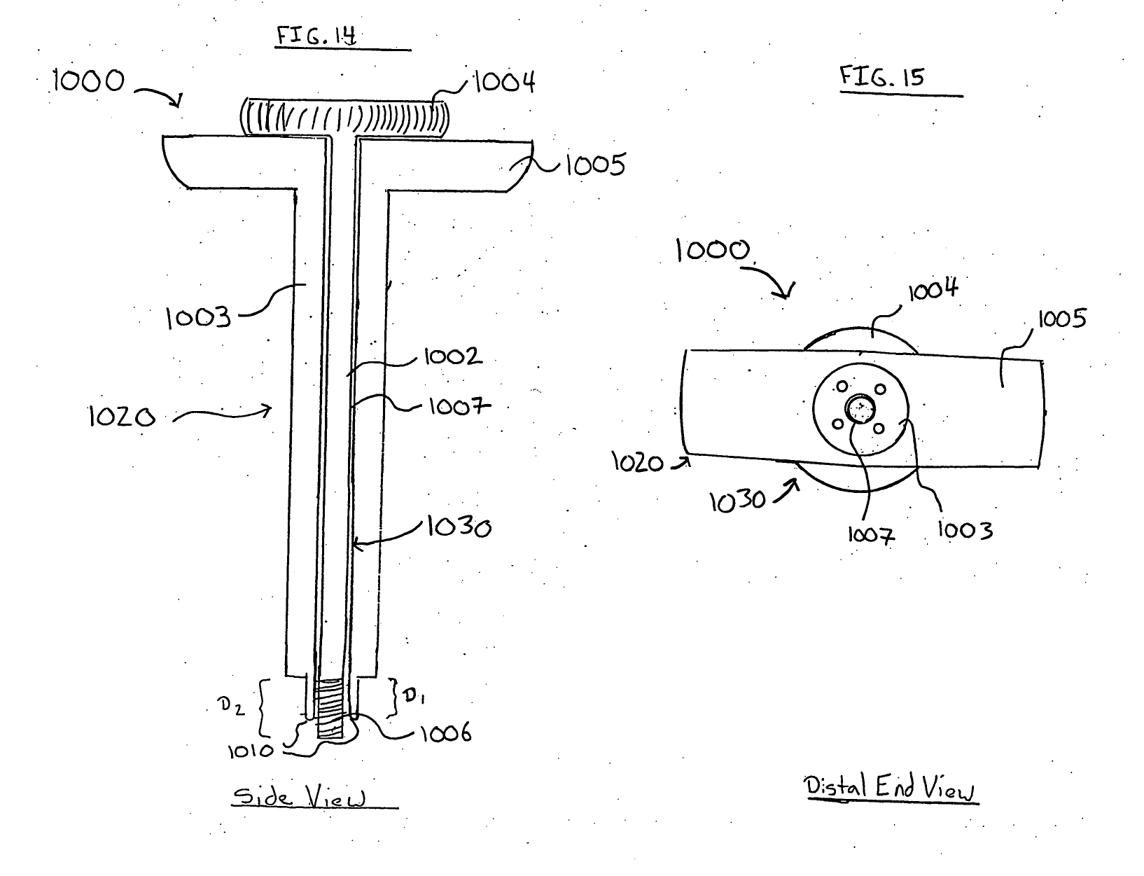

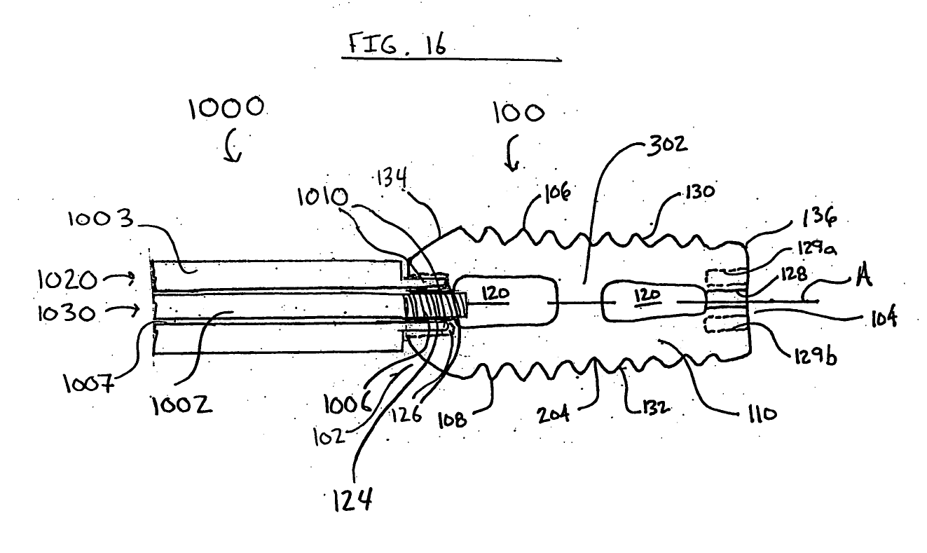

FIGS. 10-15 illustrate the tool assembly 1000 and accompanying features. The tool assembly 1000 is illustrated in FIGS. 14 and 15. The tool assembly 1000 can be manufactured with two main parts. The parts may be referred to as a first tool and a second tool in order to distinguish the two. The identification of a first tool and a second tool is not meant to describe an order, sequence, or hierarchy. The tool assembly 1000 includes an outer tool 1020, also referred to as a second tool, illustrated in FIGS. 10 and 11 and an inner tool 1030, also referred to as a first tool, illustrated in FIGS. 12 and 13.

FIGS. 10 and 11 display the side and distal end views respectively of the outer tool 1020. The outer tool 1020 has a handle 1005 at the proximal end, a shaft 1003 extending from the proximal end to the distal end, and a tooling portion configured to engage a corresponding receiving portion of the prosthetic implant. The tooling portion can include at least one pin or recess configured to exert a rotational force on the prosthetic implant. The illustrated embodiment includes at least two tooling pins 1010 extending from the distal end of the shaft 1003. The tooling pins 1010 provide means for distributing the force evenly from the outer tool 1020 to the prosthetic implant 100 during rotation of the prosthetic implant 100 in the prepared area in the spinal column, as further explained below. As shown in this example, the outer tool 1020 has four tooling pins 1010a-d.

The shaft 1003 of the outer tool 1020 has a passageway 1007 sized to allow the shaft 1002 of the inner tool 1030 to extend from the proximal end to the distal end. The shaft 1002 of the inner tool 1030 can be positioned coaxially within the shaft 1003 of the outer tool 1020 and is encircled by the shaft 1003 of the outer tool 1020. The tooling pins 1010 are adapted to engage and disengage the rotational tooling apertures 126, 129; such as the anterior rotational tooling apertures 126a-d or the posterior rotational tooling apertures 129a-d. To rotate a prosthetic implant, an operator applies a rotational force to the handle 1005. The handle 1005 transfers the rotational force through the shaft 1003 and tooling pins 1010 to the corresponding tooling apertures, for example 126a-d, of the prosthetic device.

FIGS. 12 and 13 display the side and distal end views respectively of the inner tool 1030. The inner tool 1030 has a shaft 1002 with a handle 1004 at the proximal end and a threaded portion 1006 at the distal end. The inner tool 1030 is inserted into the passageway 1007 of the outer tool 1020. The threaded portion 1006 of the inner tool 1030 engages and disengages the threads in the tooling apertures; such as the anterior tooling aperture 124, or the posterior tooling aperture 128.

FIGS. 14 and 15 display the side and distal end views respectively of the tool assembly 1000. The tool assembly 1000 has an outer tool 1020 and an inner tool 1030. The outer tool 1020 is constructed so that the inner tool 1030 engages with the outer tool 1020 such that the shaft 1002 of the inner tool 1030 extends through the passageway 1007 of the outer tool 1020 so that the threaded portion 1006 of the inner tool 1030 extends beyond the tooling pins 1010 of the outer tool 1020, as shown by D1 as the length of the tooling pins 1010 and D2 as the length of the threaded portion 1006. By way of example, the threaded portion 1006 of the inner tool 1030 extends distally beyond the distal end of the tooling pins 1010 of the outer tool 1020. This is accomplished in the current figure by having length D2 greater than length D1. This result could also be accomplished by increasing or decreasing the shaft lengths 1002 or 1003 of the inner or outer tools 1030, 1020 respectively (not shown).

The dimensions of the inner tool 1030 and the outer tool 1020 may be related such that the handle 1004 of the inner tool 1030 captures the outer tool 1020 between the handle 1004 and the prosthetic implant when the inner tool 1030 is engaged with the prosthetic implant. For example, the tooling pins 1010 of the outer tool 1020 may be seated within corresponding receiving portions of the prosthetic device. The inner tool 1030 may then be inserted through the passage of the outer tool 1020. The threaded portion 1006 of the inner tool 1030 may then engage the corresponding portion of the prosthetic device. The length of the shaft 1002 of the inner tool 1030 may be configured such that the handle 1004 of the inner tool 1030 captures and seats the outer tool 1020 to the prosthetic device when the inner tool 1030 is threaded into the prosthetic device a predetermined amount. Capturing the outer tool 1020 to the prosthetic device may ensure the rotational force can be applied to the prosthetic device without the outer tool 1020 disengaging from the prosthetic device.

FIG. 16 illustrates an exemplary engagement of the tool assembly 1000 with the anterior end 102 of the prosthetic implant 100. The tool assembly 1000 engages the anterior end 102 of the prosthetic implant 100 via engagement of the outer tool 1020 and the inner tool 1030. The anterior tooling aperture 124 is threaded to allow the threaded portion 1006 of the inner tool 1030 to selectively fasten and unfasten the tool assembly 1000 to and from the prosthetic implant 100. Further, the anterior end 102 further includes anterior rotational tooling apertures 126 to selectively engage the tooling pins 1010 of the outer tool 1020 so that the tool assembly 1000 is capable of rotating the prosthetic implant 100 upon insertion of the prosthetic implant 100 at the desired depth into the prepared area in the spinal column.

In one example of placing and rotating the prosthetic implant 100 referring to FIGS. 14-16 the inner tool 1030 is rotated via turning the handle 1004 so that the threaded portion 1006 inserts further into the threaded tooling aperture 124 or 128 the tooling pins 1010a-d are drawn into contact with the rotational tooling apertures 126a-d or 129a-d and with further rotation and alignment the tooling pins 1010a-d are secured, seated, or otherwise captured within the anterior or posterior rotational tooling apertures 126a-d or 129a-d. Once the tooling pins 1010a-d are embedded or otherwise seated within the anterior or posterior rotational tooling apertures 126a-d or 129a-d, and secured via threaded engagement of the threaded portion 1006 with the threaded tooling aperture 124 or 128, the tool assembly 1000 and the prosthetic implant 100 may be rotated within the prepared area of the spinal column as needed with a decreased risk of tooling pins disengaging or slipping from the anterior or posterior rotational tooling apertures 126a-d or 129a-d respectively. Upon completion of the rotational adjustment of the prosthetic implant, the inner tool 1030 is rotated via turning the thread control handle 1004 so that the threaded portion 1006 exits the anterior or posterior tooling aperture 124 or 128 respectively. Once the inner tool 1030 is disengaged from the prosthetic implant, the outer tool 1020 may be similarly disengaged by gently pulling the handle 1005 so that the tooling pins 1010a-d disengage from the anterior or posterior rotational tooling apertures 126a-d or 129a-d.

In another example of placing and rotating the prosthetic implant 100 where rotational torque is minimal or not necessary, the outer tool 1020 may not be necessary for the proper insertion of the prosthetic implant 100. Proper placement of the prosthetic implant 100 may be accomplished simply by using the inner tool 1020. For example, the threaded portion 1006 of the inner tool 1020 may be engaged with the corresponding portion of the prosthetic implant 100. The threaded portion 1006 can securely engage the prosthetic implant 100 to ensure the inner tool 1020 will not fall off or otherwise disengage from the implant. The prosthetic implant 100 may then be placed in the desired spinal location. The inner tool 1020 may also allow for rotation of the prosthetic implant 100. The inner tool 1020 can be unthreaded from the prosthetic implant 100 to disengage the inner tool 1020 once the implant is properly positioned.

In another example of placing and rotating the prosthetic implant 100 where rotational torque is minimal or not necessary, the inner tool 1030 may not be necessary for the proper insertion of the prosthetic implant 100. Proper placement of the prosthetic implant 100 may be accomplished simply by using the outer tool 1020 by manually aligning and inserting the tooling pins 1010a-d into the rotational tooling apertures 126a-d or 129a-d until the tooling pins 1010a-d are embedded within the anterior or posterior rotational tooling apertures 126a-d or 129a-d. The prosthetic implant 100 may be merely placed or rotated within the prepared area of the spinal column as needed. Upon completion of the rotational adjustment and/or placement of the prosthetic implant 100, the outer tool 1020 may be disengaged from the prosthetic implant 100 by pulling the handle 1005 so that the tooling pins 1010a-d disengage from the anterior or posterior rotational tooling apertures 126a-d or 129a-d.

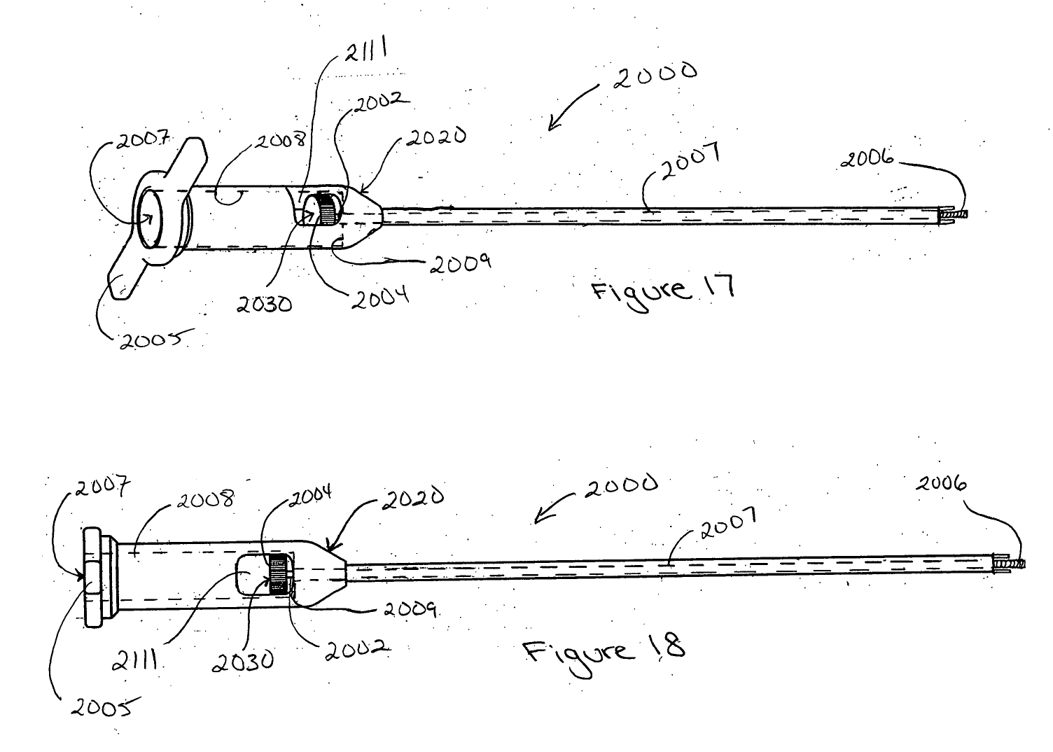

As discussed above, tool assembly 1000 comprises an inner tool 1030 and an outer tool 1020 that when assembled engage one another in a manner that results in handle 1004 resting on the surface of handle 1005. FIGS. 17 and 18 illustrate an alternative configuration whereby tool assembly 2000 comprises an inner tool 2030 and an outer tool 2020 that still function as described previously; however, in this configuration handle 2004 is not seated on handle 2005 as described previously. In this alternative configuration, outer tool 2020 has an axial passageway 2007 extending longitudinally therethrough. The proximal end of passageway 2007 has a larger diameter end portion 2008, which results in an annular seating surface shoulder 2009 extending radially outward from passageway 2007 for the bearding of handle 2004. Outer tool 2020 also has at least one opening or window 2111 located at shoulder 2009.

When assembled, inner tool 2030 is positioned coaxially within outer tool 2020 such that handle 2030 captures the outer tool 2020 between annular seating surface shoulder 2009 and the prosthetic implant when the inner tool 2030 is engaged with the prosthetic implant (not shown). Window 2111 is positioned so that handle 2004, when operationally positioned, can be accessed from one or two sides and easily turned thus allowing the threaded portion 2006 of shaft 2002 to engage or disengage prosthetic implant 100. An advantage of recessing handle 2004 within the body of outer tool 2020 is that handle 2005 can be struck without directly striking handle 2005.

In yet another example of inserting the prosthetic implant 100, the prosthetic implant 100 is inserted into the posterior side of the spinal column and the posterior side of the patient. Therefore, the tool assembly 1000 and the outer tool 1020 is releasably secured or fastened to the posterior rotational tooling apertures 129a-d. Insertion areas are prepared in the spinal column between the vertebrae, and the prosthetic implant 100 is inserted into the spinal column with the anterior end 102 being inserted into the prepared area of the spinal column. The prosthetic implant is inserted to the desired depth in the prepared area with the top side serrations 130 and the bottom side serrations 132 engaging the bony tissue of the adjacent vertebrae, such as 702 and 704 as shown in FIGS. 7 and 8. The tool assembly 1000 is then unfastened from the prosthetic implant 100 by unsecuring or unfastening, and in the example shown, the outer tool 1020 may be disengaged from the prosthetic implant 100 by pulling the handle 1005 so that the tooling pins 1010a-d disengage from the posterior rotational tooling apertures 129a-d. In this example, the prosthetic implant 100 was not rotated in the prepared area in the spinal column and, therefore, the inner tool 1030 may be removed from the insertion tool 1000 prior to the placement of the prosthetic implant 100 into the spinal column, since it is not needed to rotate the implant.

In a final example, the prosthetic implant 100 is inserted from the anterior side of the spinal column and the anterior side of the patient. In this example, the threaded portion 1006 is releasably secured or fastened to the anterior tooling aperture 124 and the tooling pins 1010a-d are slidably engaged with the anterior rotational tooling apertures 126a-d. The prosthetic implant 100 is then inserted into the prepared area in the spinal column in a side ways orientation so that the top side serrations 130 and the bottom side serrations 132 are latitudinally oriented when the prosthetic implant 100 is inserted into the prepared area of the spinal column. After the prosthetic implant 100 is inserted to the desired depth in the spinal column by use of the tool assembly 1000, the prosthetic implant 100 is rotated ninety degrees by the surgeon holding the thread control handle 1004 in one position but by rotating the handle 1005 ninety degrees so that the tooling pins 1010 exert a rotational force to the prosthetic implant 100 through the anterior rotational tooling apertures 126. During rotation of the prosthetic implant 100, the top side serrations 130 and bottom side serrations 132 purchase, or engage the bone tissue of the adjacent vertebrae, such as 702 and 704 as shown in FIG. 8. After the prosthetic implant 100 is rotated to its proper final orientation, the thread control handle 1004 is turned so that the threaded portion 1006 exits the anterior tooling aperture 124, the outer tool 1020 is then backed away from the prosthetic implant 100 so that the tooling pins 1010 disengage the anterior rotational tooling apertures 126.

The prosthetic implant of the present disclosure is made of a biocompatable material, such as poly-ether-acetone, poly-ether-ether-ketone (PEEK), carbon fiber, ceramic, titanium, or some other material that is biocompatable.

The tool assembly of the present disclosure is made of a material capable of being sterilized via autoclave, such as carbon fiber, ceramic, titanium, stainless steel, or some other material that is durable and capable of being sterilized for medical applications. In an exemplary embodiment the inner tool extends beyond the distal end of the tooling pins of the outer tool by having the length of the threaded portion D2 be greater than length of the tooling pins D1 such that D2 is approximately 10 mm and D1 is approximately 5 mm. This increased length results in the inner tool having more than enough threads to keep engaging the prosthetic implant until the tooling pins are fully embedded with the rotational tooling apertures of the prosthetic implant. This result could also be accomplished in multiple other ways including, but not limited to increasing or decreasing the shaft lengths of the inner or outer tools.

The prosthetic implant disclosed herein provides the surgeon the option of placing the prosthetic implant from the anterior side or the posterior side of the spinal column, and allows the surgeon the option of inserting the prosthetic implant in an orientation without requiring rotation of the prosthetic implant after the implant is inserted to the desired depth in the spinal column. In the alternative, the disclosed implant allows the surgeon the option of inserting the prosthetic implant in a side ways orientation and then rotating the prosthetic implant ninety degrees to its final orientation.

It should be understood that the prosthetic implant disclosed may be modified while still providing the benefits and features of the disclosed herein. For example, the right side portion and the left side portion of the prosthetic implant may include more than one rib each and, further, the top side and the bottom side of the implant may further include rib portions as well. Further, the disclosed implant may be modified so as to provide other geometric shapes as viewed from one end, such as the end view shown in FIGS. 5 and 6. For example, the implant may be hexagonal or octagonal in shape as viewed from one end, which may provide additional surface area for the implant to oseointegrate with the bone tissue.

Although this disclosure has been shown and described with respect to detailed embodiments, those skilled in the art will understand that various changes in form and detail may be made without departing form the scope of the claimed disclosure.

What is claimed is:

1. A prosthetic implant, comprising:
an anterior end;
a posterior end located opposite of the anterior end, the anterior end being larger than the posterior end;
a left side portion and an opposite right side portion separating the anterior end from the posterior end; and
a top side and an opposite bottom side on each of the left side portion and the right side portion, the top and bottom sides of each left side portion and right side portion having serrations.

2. The prosthetic implant of claim 1, wherein the left side portion and the right side portion define a longitudinal slot.

3. The prosthetic implant of claim 2, wherein the left side portion and the right side portion define a longitudinal through slot.

4. The prosthetic implant of claim 1, further comprising means for accepting an insertion tool for positioning the prosthetic implant.

5. The prosthetic implant of claim 1, wherein the anterior end and the posterior end each define a tooling aperture.

6. The prosthetic implant of claim 1, wherein the anterior end defines an anterior tooling aperture, and wherein the posterior end defines a posterior tooling aperture.

7. The prosthetic implant of claim 6, wherein the anterior end further comprises anterior rotational tooling apertures for the placement of the prosthetic implant.

8. The prosthetic implant of claim 7, wherein the posterior end further comprises posterior rotational tooling apertures for the placement of the prosthetic implant.

9. The prosthetic implant of claim 8, wherein the right side portion further comprises a right side rib defining a right side anterior aperture and a right side posterior aperture.

10. The prosthetic implant of claim 9, wherein the left side portion further comprises a left side rib defining a left side anterior aperture and a left side posterior aperture.

11. The prosthetic implant of claim 1, wherein the anterior end further comprises rounded edges.

12. The prosthetic implant of claim 11, wherein the posterior end further comprises rounded edges.

13. The prosthetic implant of claim 1, wherein the serrations include radius edges.

14. The prosthetic implant of claim 1, wherein the prosthetic implant comprises a biocompatible material.

15. The prosthetic implant of claim 14, wherein the biocompatible material comprises at least one material chosen from the group comprising poly-ether-acetone, poly-ether-ether-ketone (PEEK), carbon fiber, and ceramic.

16. A prosthetic implant, comprising:
an anterior end;
a posterior end located opposite of the anterior end; and
left and right side portions separating the anterior end from the posterior end, each of the left and right side portions having top end serrations and substantially opposing bottom end serrations, the serrations comprising a plurality of peaks, wherein at least one pair of adjacent peaks are separated by a lower radius.

17. The prosthetic implant of claim 16, wherein the anterior end is larger than the posterior end.

18. The prosthetic implant of claim 16, wherein the anterior end comprises:
an interface portion configured to releasably engage a first portion of an implant tool; and
a receiving portion configured to engage a second portion of the implant tool, and configured to receive a rotational force applied by the second portion of the implant tool.

19. The prosthetic implant of claim 16, wherein the anterior end includes a threaded hole for receiving an implant placement tool, and further includes at least one tooling aperture to receive a rotational force.

20. The prosthetic implant of claim 16, wherein the anterior end comprises substantially rounded edges.

21. The prosthetic implant of claim 16, wherein each of the anterior end and the posterior end includes a threaded hole for receiving an implant placement tool, and at least one of the anterior end and the posterior end includes a plurality of tooling apertures configured to receive a rotational force.

22. A method of placing a prosthetic implant, the method comprising:
providing a prosthetic implant, the implant comprising:
an anterior end including an anterior tooling aperture and anterior rotational tooling apertures;
a posterior end being located opposite of the anterior end, the anterior end being larger than the posterior end, and wherein the posterior end includes a posterior tooling aperture and posterior rotational tooling apertures;
a left side portion and an opposite right side portion separating the anterior end from the posterior end; and
a top side and an opposite bottom side on each of the left side portion and the right side portion, the top and bottom sides of each left side portion and right side portion having serrations;
creating a void in either the anterior or the posterior side of a vertebrae in a spinal column; and
inserting the posterior end of the prosthetic implant into the vertebrae first if the void is created from the anterior side of the vertebrae, or inserting the anterior end of the prosthetic implant into the vertebrae first if the void is created from the posterior side of the vertebrae.

23. The method of placing a prosthetic implant of claim 22, further comprising rotating the prosthetic implant in the vertebrae.

24. The method of placing the prosthetic implant of claim 22, wherein if the prosthetic implant is placed through the posterior side of the vertebrae, the prosthetic implant is inserted into the vertebrae at the desired depth and then rotated substantially ninety degrees so that the serrations are longitudinally oriented.

25. The method of inserting the prosthetic implant of claim 22, wherein if the prosthetic implant is placed through the anterior side of the vertebrae, the prosthetic implant is inserted into the vertebrae at the desired depth with the serrations being longitudinally oriented.

26. The method of placing the prosthetic implant of claim 22, further comprising:
providing tooling for placing the prosthetic implant, wherein the tooling has means for selectively fastening and unfastening to either the anterior end or the posterior end of the prosthetic implant.

27. The method of placing the prosthetic implant of claim 26, wherein the tooling further comprises a threaded portion for selectively fastening and unfastening to the prosthetic implant, and the tooling further comprises a tooling pin portion that selectively engages or disengages the prosthetic implant for rotating the prosthetic implant after insertion.

28. The method of placing the prosthetic implant of claim 26, wherein the tooling further comprises a tooling pin portion for selectively engaging and disengaging the prosthetic implant, for placing the prosthetic implant after insertion.

29. The method of placing the prosthetic implant of claim 26, wherein the tooling further comprises a tooling pin portion for selectively engaging and disengaging the prosthetic implant, for rotating the prosthetic implant after insertion.

30. The method of placing the prosthetic implant of claim 26, wherein the tooling further comprises a tooling pin portion for selectively engaging and disengaging the prosthetic implant, for placing and rotating of the prosthetic implant after insertion.

31. A prosthetic implant tool assembly, the tool assembly comprising:
a first tool having an interface portion configured to releasably engage a prosthetic implant; and
a second tool having a tooling portion configured to engage a receiving portion of the prosthetic implant while the first tool is engaged with the prosthetic implant, and further configured to apply a rotational force to the prosthetic implant via the receiving portion.

32. The tool assembly of claim 31, wherein the first tool comprises an inner shaft having the interface portion positioned substantially on one end, and wherein the second tool comprises an outer shaft having at least a portion that substantially encircles at least a portion of the inner shaft of the first tool.

33. The tool assembly of claim 32, wherein the portion of the inner shaft is coaxial with, and extends through the portion of the outer shaft.

34. The tool assembly of claim 32, wherein the interface portion comprises a threaded portion.

35. The tool assembly of claim 31, wherein the tooling portion of the second tool comprises a plurality of tooling pins.

36. The tool assembly of claim 31, wherein the second tool further comprises:
a shaft having the tooling portion positioned substantially at one end; and
a handle positioned on the shaft substantially opposite the tooling portion, and configured to receive the rotational force.

37. The tool assembly of claim 31, wherein the first tool comprises a handle substantially opposite the interface portion.

38. The tool assembly of claim 37, wherein the handle is configured to capture the tooling portion of the second tool with the receiving portion of the prosthetic implant when the interface portion of the first tool is engaged with the prosthetic implant.

39. A prosthetic implant tool assembly, the tool assembly comprising:
an inner shaft having a threaded end portion configured to engage a portion of a prosthetic implant;
an inner shaft handle positioned substantially opposite the threaded end portion;
an outer shaft substantially coaxial to the inner shaft and configured to receive the inner shaft, the outer shaft captured between the inner shaft handle and the prosthetic implant when the threaded portion of the inner shaft is engaged with the portion of the prosthetic implant;
a plurality of tooling pins positioned substantially at an end of the outer shaft;
a handle positioned along the outer shaft substantially opposite the plurality of tooling pins, and configured to receive a rotational force and couple the rotational force, via the outer shaft, to the plurality of tooling pins.
